(19) United States
(12) Patent Application Publication
(10) Pub. No.: US 2017/0027760 A1
(43) Pub. Date: Feb. 2, 2017

(54) Rhinoderm

(71) Applicant: Myungsoon Ji, Oak Harbor, WA (US)

(72) Inventor: Myungsoon Ji, Oak Harbor, WA (US)

(21) Appl. No.: 14/816,059

(22) Filed: Aug. 2, 2015

(51) Int. Cl. A61F 13/02; A61F 5/30

(52) U.S. Cl. CPC A61F 13/022; A61F 5/30; A61F 13/0226; A61F 13/025; A61F 2013/00217

(57) ABSTRACT

A flexible nose bandage is disclosed. The bandage is a surgical bandage for nose, wherein the bandage is comprised of foam, cloth, porous flexible material, and woven adhesive material or the like, which includes opposing infra orbital portions, opposing side arms, and a connecting bridge to place the bandage securely at its position. The bandage is further comprised of components like non-stick gauze sponge pads, to be applied over the wound, and waterproof edges made of thin film tegaderm to cover the entire outer surface of the dressing.

### CROSS REFERENCE TO RELATED APPLICATION

Not Applicable

### FEDERALLY SPONSORED RESEARCH AND DEVELOPMENT

Not Applicable

### MICROFICHE APPENDIX

Not Applicable

### BACKGROUND OF THE INVENTION

Field of the Invention

The present invention relates to the field of medical dressing aids, specifically to surgical dressings. More particularly, the present invention relates to surgical dressings for nose wounds. The present invention also relates to surgical pressure dressings, and, more particularly to a pressure dressing for use with nose injuries that require pressure on the injured area.

Background of the Invention

As per the recorded data, each year in the US, nearly five million people are treated for skin cancer. Each year, there are more new cases of skin cancer than the combined incidence of cancers of the breast, prostate, lung, and colon; treatment of non-melanoma skin cancers increased by nearly 77 percent between 1992 and 2006. Over the past three decades, more people have had skin cancer than all other cancers combined, wherein one in five Americans will develop skin cancer in the course of a lifetime. Young men account for just 40 percent of melanoma cases, but more than 60 percent of melanoma deaths. From ages 15-39, men are 55 percent more likely to die of melanoma than women in the same age group. An estimated 43,890 new cases of invasive melanoma in men and 32,210 in women will be diagnosed in the US in 2014; an estimated 6,470 men and 3,240 women in the US will die from melanoma in 2014. The disadvantages associated with surgical dressings includes trauma with sub-sequential bleeding upon removal, reduced comfort, skin damage and possible pressure hurting, possible obstructive sleep apnea syndrome, and other side effects. Some patients cannot cover up the wounds, which causes infections and delays the scab-forming healing process.

Disposable bandages, particularly of the self-adhesive type, are widely used to cover and protect superficial wounds. In many cases the conventional form of such bandages is adequate. However, in some instances, flow of blood or other fluids from wounds, such as cuts and abrasions, is more serious than can be stopped by these bandages.

The general form of disposable bandage generally includes a pad for positioning on and over the wound that is generally sterile and non-sticking. This is backed by a piece of pressure adhesive material, which holds the pad on the wound. The shape can vary, being square, elongate, circular, or any other shape. In some circumstances, a separate pad is placed over the wound, and then a piece of pressure adhesive material placed over the pad and into contact with the patient's skin to hold the pad in place.

Limited pressure can be applied with the conventional bandage or dressing. Some pressure can be applied when the wound is on a rounded part of a body, such as a finger, arm, or other well-rounded part. On a flat or only slightly rounded part of the body, such as the back, chest, top part of legs, and similar parts, minimal to no pressure can be applied to the wound.

Various attempts have been made to provide pressure. In some prior arts, pads are provided for localizing pressure. One of the prior art discloses a bandage that is not of the self-adhesive type. This bandage is wound round an area, or, similar to normal bandages, over the pad. Another prior art discloses an adhesive tape that has a section of resilient foam, which is held partially compressed by the tape. Such a pad is bulky, which can make it awkward to wear such a dressing. A non-stick sterile layer must be provided over the pad to prevent it from adhering to the wound. Similarly, an adhesive tape holds down a section of resilient foam in some other prior art. Others provide a strap for wrapping round a limb, with a pad of resilient members held against the limb. This cannot be used as a localized dressing. Some have an inelastic strut for positioning over a wound, with an expansible pouch. The pouch is inflatable by a pump. Alternatively, the pouch can contain a powder and a frangible capsule containing liquid. The liquid, when released, reacts with the powder to form a gas, which inflates the pouch. This is a complex and relatively expensive bandage.

The present invention provides a bandage or dressing that can be applied to a wound, and will apply pressure to the wound.

Particularly, the invention provides a disposable bandage or dressing that has a pad for positioning over and on the wound, a flexibly resilient arcuate member for positioning over the pad, the arcuate member having a convex surface facing towards the pad, an adhesive layer over the plastic member and extending beyond the plastic member, and the pad for attachment to the body of a patient.

In the broadest concept, a dressing can be comprised of separate pads and an arcuate plastic member, the two, when in position, are held in place by the adhesive material. Conveniently, a bandage will be comprised of a pad, plastic member, and adhesive material that are pre-assembled together, with a removable covering over the pad.

The traditional compress-type bandage consists of a sterile absorbent pad, sewn or otherwise attached to a length of surgical gauze. It is simple, cheap, and effective, but has some limitations. Care must be exercised in applying the gauze tails, so as not to cause constrictions that may restrict blood flow. The bandage must normally be removed in order to apply medication to the wound, and since the pads tend to stick to wounds, removal of the bandage may break or pull away protective scabs. Whilst the bandages may be sealed in packages to protect sterility, it is often difficult to open such packages and unfold the product for use without endangering the sterility of the pad, particularly when working under adverse conditions.

### SUMMARY

The present invention provides an absorbent, self-adhering elastic wound dressing, as well as a method of applying a compressive dressing to a wound. The elastic wound dressing of the present invention includes a porous, self-adhering elastic substrate that does not adhere to clothing, hair, or skin, and which has a permanent compressive force when extended that is sufficient to hold the dressing in place and to provide a therapeutic effect to the wound, an absorbent layer covering at least a portion of the self-adhering substrate, and a means adapted to flexibly bond the self-adhering substrate to the absorbent layer.

It is, accordingly, a principal objective of the invention to provide an improved means for anchoring a nasal surgical bandage on a patient.

Another objective of the invention is to provide a preformed nasal surgical bandage that can be applied in only a few seconds and can be left in place for prolonged periods of time without causing irritation to skin tissues of the nose.

Yet another objective of the invention is to provide a surgical bandage for securing a length of medical bandage to the body of a patient that is comprised of a pre-shaped adhesive patch that, because of its shape, readily conforms to the surfaces to which attachment is to be made.

An objective of the present invention is to remove the above-mentioned defects or shortcomings involved in the prior art materials, and to provide a wound dressing that is useful for absorbing exudate from a wound to prevent the exudate remaining between the dressing material and the surface of the wound, permitting tissues to develop, and capable of protecting against the invasion of foreign bacteria with the result of promoting therapeutic effects.

Another objective of the present invention is to provide a wound dressing that is capable of preventing bacterial infection of the wound and transudation of body fluids, thus enabling fast healing of the wound.

A further objective of the present invention is to provide a wound dressing of a multilayer structure that contains a therapeutic agent, so that the above-mentioned therapeutic effects can be displayed satisfactorily.

It is also an objective of the present invention to provide a formable pressure dressing for use in the treatment of nose injuries that require pressure to prevent the re-accumulation of blood in the injured area.

It is another objective to provide a pressure dressing that is formable to the particular shape or size of the patient's nose.

It is yet another objective to provide a pressure dressing that is easily removable for periodic examination and cleaning of the nose, and then is easily replaceable for continued use.

These and other objectives and advantages of the invention will become apparent to those skilled in the art from the following detailed description of a preferred embodiment, especially when considered in conjunction with the accompanying drawings.

### BRIEF DESCRIPTION OF THE DRAWINGS

To further clarify various aspects of some example embodiments of the present invention, a more particular description of the invention will be rendered by reference to specific embodiments thereof, which are illustrated in the appended drawing. It is appreciated that the drawing depicts only illustrated embodiments of the invention, and is, therefore, not to be considered limiting of its scope. The invention will be described and explained with additional specificity and detail through the use of the accompanying drawing, in which:

FIG. 1 depicts a perspective view of the invention, depicting a surgical bandage for the nose

### DETAILED DECRIPTION OF THE INVENTION

The embodiments of the present disclosure, described below, are not intended to be exhaustive or to limit the disclosure to the precise forms disclosed in the following detailed description. Rather, the embodiments are chosen and described so that others skilled in the art may appreciate and understand the principles and practices of the present disclosure.

The general purpose of the present invention is to provide a nose bandage that is one piece and provides secure coverage in either unilateral or bilateral nasal occlusions. Specifically, the nose bandage can be used to retain nasal packing or other nasal occlusion devices. The nose bandage also provides nasal-facial protective coverings for the care of external nasal-facial injuries.

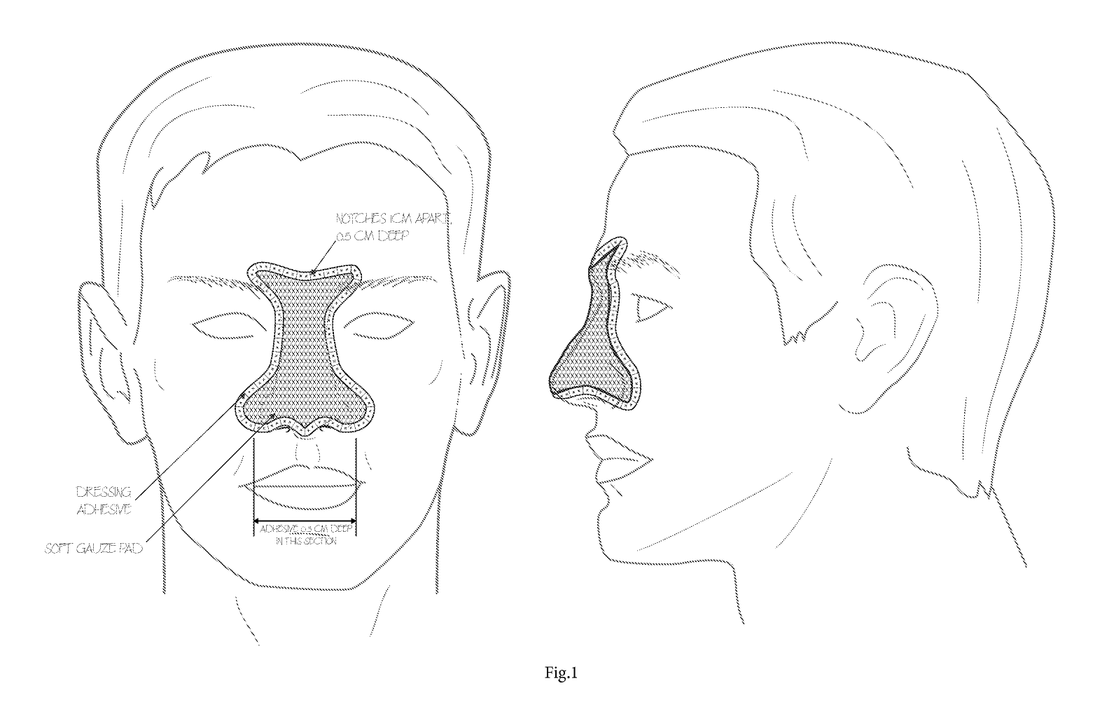

In FIG. 1, the present invention discloses a surgical bandage for the nose, wherein the length of the top part of the nose (right between the eyes) should be 2.5 cm. The notches, as marked should be 1 cm apart and 0.5 cm deep. The adhesive part of the dressing is stretchy, while the soft gauze pad is included to increase flexibility and be thick enough to give the wound pressure. More importantly, the primary function of these dressings is to create pressure to control bleeding issues.

Accordingly, the gauze pad in this case should be soft enough to meet the anatomy of the nose. Furthermore, additional non-adherent pads or cotton balls can also be placed where dressing fails to apply adequate amount of pressure.

According to one embodiment of the present invention, there is provided a nose bandage of foam, cloth, porous flexible material, woven adhesive material, or the like, which includes opposing infra orbital portions, opposing side arms, and a connecting bridge there between. The geometrical configuration of the infra orbital portions include parallel orbital sides, and outwardly curved opposing sides. The opposing side arms include parallel arm sides and outwardly curved opposing sides. Two perforated holes are provided as nasal passages in the center-mid portion of the opposing side arms, and there is an adhesive coating with a protective strip provided on one side of the bandage for securing over and about the skin of the infra orbital portion of the face.

In a preferred embodiment, the present invention is made of components like non-stick gauze sponge pad, which is a non-adherent pad that is applied over the wound, and water proof edges, which is a thin film called tegaderm to cover the entire outer surface of the dressing, not just the edges and adhesive. In addition to this, the non-stick gauze pad protects the wound and the pads by not sticking to the wound.

Although specific embodiments have been illustrated and described herein, it will be appreciated by those of ordinary skill in the art that any arrangement that is calculated to achieve the same purpose may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

I claim:

1. A nose bandage comprised of:
(a) opposing infra orbital portions;
(b) opposing side arms;
(c) connecting bridge;
(d) non-stick soft gauze pad; and
(e) adhesive ends.

2. A nose bandage, as claimed in claim 1, wherein the geometrical configuration of said opposing infra orbital portions includes parallel orbital sides and outwardly curved opposing sides.

3. A nose bandage, as claimed in claim 1, wherein said opposing side arms include parallel arm sides and outwardly curved opposing sides, and two perforated holes to provide nasal passages in the center mid portion of the opposing side arms.

4. A nose bandage, as claimed in claim 1, wherein said adhesive ends coating with a protective strip is provided on one side of bandage for securing over and about the skin of the infra orbital portion of the face.

5. A nose bandage, as claimed in claim 1, wherein the non-stick said soft gauze sponge pad provided is a non-adherent pad applied over wound with waterproof edges.

6. A nose bandage, as claimed in claim 5, wherein said waterproof edges are made of a thin film called tegaderm to cover the entire outer surface of the dressing.

7. A nose bandage, as claimed in claim 1, wherein said non-stick soft gauze sponge pad provided is a non-adherent pad applied over wound with said waterproof edges.

8. A nose bandage, as claimed in claim 5, wherein said waterproof edges are made of a thin film called tegaderm to cover the entire outer surface of the dressing.
